(19) United States
(12) Patent Application Publication
(10) Pub. No.: US 2023/0062268 A1
(43) Pub. Date: Mar. 2, 2023

(54) SYSTEM AND METHOD FOR FATIGUE RESPONSE PREDICTION

(71) Applicant: Siemens Industry Software NV, Leuven (BE)

(72) Inventors: Lucia Mirabella, Plainsboro, NJ (US); Elena Arvanitis, Somerville, NJ (US); Dehao Liu, Marietta, GA (US); Nicolas Lammens, Herent (BE); Hunor Erdelyi, Oud-Heverlee (BE); Christoph Ernst Ludwig, Munich (DE)

(21) Appl. No.: 17/760,367

(22) PCT Filed: Feb. 25, 2020
(86) PCT No.: PCT/US2020/019691

§ 371 Date: Aug. 9, 2022

(51) Int. Cl. G06F 30/27; G06F 30/23

(52) U.S. Cl. CPC G06F 30/27; G06F 30/23

(57) ABSTRACT

A computer-implemented method is provided for predicting a fatigue response of a material. The method includes receiving a user input specifying one or more surface roughness parameters that characterize a surface of a material for which fatigue life is to be predicted. The method further includes generating at least one realistic virtual surface profile from the specified one or more surface roughness parameters. The method further includes predicting fatigue life of the material in dependence of a stress field applied to the generated virtual surface profile. In accordance with specific embodiments, the prediction of the fatigue life may be carried out using finite element analysis based simulations, machine learning methods, or combinations thereof.

### BACKGROUND

### 1. Field

The present disclosure relates, in general, to the field of material fatigue response prediction. Embodiments of the present disclosure are applicable, for example, for predicting fatigue life of additively manufactured parts from surface roughness parameters.

### 2. Description of the Related Art

Industrial use of additive manufacturing (AM) is becoming ever so common for manufacturing complex parts with high repeatability and performance. Use of AM technologies save time and resources, which are one of the advantages of AM over conventional manufacturing techniques. However, parts fabricated by AM generally have poor surface roughness. Moreover, surface roughness varies from region to region within a component based on geometry (e.g. overhang angle).

Surface quality of a part may have a significant impact on the fatigue life of the part. Surface preparation and machining techniques that reduce surface roughness have been successfully employed to extend fatigue lives of parts that are subjected to cyclic stresses. While such methods may be applicable generally to external surfaces of additively manufactured parts, the internal “as-built” surfaces of complex geometry parts may prove more challenging to modify.

Techniques have been developed for assessing the influence of surface roughness on fatigue performance. However, state of the art techniques require expensive and time-consuming experimentation and/or heavy computational resources.

### SUMMARY

Briefly, aspects of the present disclosure are directed to techniques for prediction of fatigue response based on surface roughness that address at least some of the technical challenges mentioned above.

According to an aspect of the present disclosure, a computer-implemented method is provided for predicting a fatigue response of a material. The method comprises receiving a user input specifying one or more surface roughness parameters that characterize a surface of a material for which fatigue life is to be predicted. The method further comprises generating at least one realistic virtual surface profile from the specified one or more surface roughness parameters. The method further comprises predicting fatigue life of the material in dependence of a stress field applied to the generated virtual surface profile.

In accordance with specific non-limiting embodiments disclosed herein, the virtual surface profile may be generated utilizing machine learning based generative models, frequency pattern matching, Autoregressive Moving Average (ARMA) models, or combinations thereof. Furthermore, in accordance with specific non-limiting embodiments disclosed herein, the prediction of the fatigue life may be carried out utilizing finite element analysis based simulations, machine learning methods, or combinations thereof.

Other aspects of the present disclosure implement features of the above-described method in computing systems and computer program products.

### BRIEF DESCRIPTION OF THE DRAWINGS

The foregoing and other aspects of the present disclosure are best understood from the following detailed description when read in connection with the accompanying drawings. To easily identify the discussion of any element or act, the most significant digit or digits in a reference number refer to the figure number in which the element or act is first introduced.

FIG. 1 is a block diagram of an example of a computing system wherein aspects of the present disclosure may be implemented

FIG. 2 is a flowchart broadly illustrating a method for predicting fatigue response based on surface roughness.

FIG. 3 illustrates a 1D surface curve showing microscopic asperities.

FIG. 4 is a flowchart illustrating an example method for generating a virtual surface profile by a machine learning based generative model.

FIG. 5 is a flowchart illustrating an example method for generating a virtual surface profile by an Autoregressive Moving Average (ARMA) model.

FIG. 6 is a flowchart illustrating an example method for generating a virtual surface profile based on frequency pattern matching.

FIG. 7 is a flowchart illustrating a method for predicting fatigue response based on surface roughness according to a first example implementation.

FIG. 8 is a flowchart illustrating a method for predicting fatigue response based on surface roughness according to a second example implementation.

FIG. 9 is a flowchart illustrating a method for predicting fatigue life based on surface roughness according to a third example implementation.

### DETAILED DESCRIPTION

Aspects of the present disclosure relate to prediction of fatigue response based on surface roughness. Surface roughness is generally poor for parts fabricated by additive manufacturing (AM), which may significantly affect the fatigue performance of such parts. To assess surface roughness, one can perform surface height measurements, which may be expensive to perform and/or not accessible to all users. To assess the fatigue life of a part, a known approach is to not only quantify the surface roughness, but also generate a huge database of fatigue data corresponding to the different surface conditions. Generating such database currently is done through experiments, meaning that a large number of test specimens need to be manufactured with different surface conditions and then tested. Such an experimental campaign may be time consuming and expensive. Moreover, one needs to repeat it for different AM machines, process settings and powders. An alternative to the experimental approach is to predict the effect of surface roughness on fatigue life through simulation techniques. However, such techniques may be computationally intensive and time consuming. Furthermore, such techniques also require surface measurements, which may be difficult and expensive to perform.

The techniques disclosed herein address at least the above described problems by automatically generating a realistic virtual surface profile based on user-specified surface roughness parameters and predicting the corresponding fatigue property in dependence of a stress field applied to the virtual surface profile. The proposed techniques rely on finite element analyses (FEA), advanced machine learning (ML) methods, or combinations thereof. Using the proposed techniques, one can obtain in an efficient numerical way the fatigue property corresponding to any user defined surface roughness parameters, thereby avoiding expensive and time-consuming experimental methods. Aspects of present disclosure may be embodied in a computer-aided engineering (CAE) or computer-aided manufacturing (CAM) package.

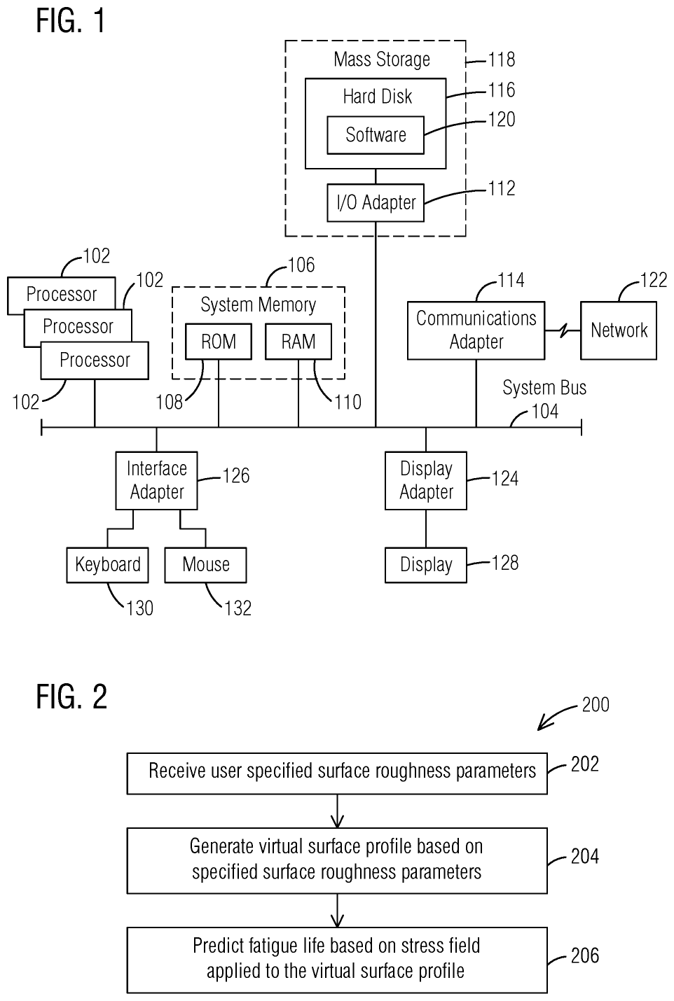

Turning now to FIG. 1, a computing system 100 is generally shown wherein aspects of the present disclosure may be implemented. The computing system 100 can be an electronic, computer framework comprising and/or employing any number and combination of computing devices and networks utilizing various communication technologies. The computing system 100 may be easily scalable, extensible, and modular, with the ability to change to different services or reconfigure some features independently of others. The computing system 100 may be, for example, a server, desktop computer, laptop computer, tablet computer, or smartphone. In some examples, computing system 100 may be a cloud computing node.

Computing system 100 may be described in the general context of computer executable instructions, such as program modules, being executed by a computing system. Generally, program modules may include routines, programs, objects, components, logic, data structures, and so on that perform particular tasks or implement particular abstract data types. Computing system 100 may be practiced in distributed cloud computing environments where tasks are performed by remote processing devices that are linked through a communications network. In a distributed cloud computing environment, program modules may be located in both local and remote computing system storage media including memory storage devices.

As shown in FIG. 1, the computing system 100 has one or more processors 102, which may include, for example, one or more central processing units (CPU), graphics processing units (GPU), or any other processor known in the art. The processors 102 can be a single-core processor, multi-core processor, computing cluster, or any number of other configurations. The processors 102, also referred to as processing circuits, are coupled via a system bus 104 to a system memory 106 and various other components. The system memory 106 can include a read only memory or ROM 108 and a random access memory or RANI 110. The ROM 108 is coupled to the system bus 104 and may include a basic input/output system (BIOS), which controls certain basic functions of the computing system 100. The RAM 110 is read-write memory coupled to the system bus 104 for use by the processors 102. The system memory 106 provides temporary memory space for operations of said instructions during operation. The system memory 106 can include random access memory (RAM), read only memory, flash memory, or any other suitable memory systems.

The computing system 100 comprises an I/O adapter 112 (input/output adapter) and a communications adapter 114 coupled to the system bus 104. The I/O adapter 112 may be a small computer system interface (SCSI) adapter that communicates with a hard disk 116 and/or any other similar component. The I/O adapter 112 and the hard disk 116 are collectively referred to herein as a mass storage 118.

Software 120 for execution on the computing system 100 may be stored in the mass storage 118. The mass storage 118 is an example of a tangible storage medium readable by the processors 102, where the software 120 is stored as instructions for execution by the processors 102 to cause the computing system 100 to operate, such as is described herein below with respect to the various figures. Examples of computer program product and the execution of such instruction is discussed herein in more detail. The communications adapter 114 interconnects the system bus 104 with a network 122, which may be an outside network, enabling the computing system 100 to communicate with other such systems. In one embodiment, a portion of the system memory 106 and the mass storage 118 collectively store an operating system, which may be any appropriate operating system, to coordinate the functions of the various components shown in FIG. 1.

Additional input/output devices are shown as connected to the system bus 104 via a display adapter 124 and an interface adapter 126. In one embodiment, the I/O adapter 112, the communications adapter 114, the display adapter 124 and the interface adapter 126 may be connected to one or more I/O buses that are connected to the system bus 104 via an intermediate bus bridge (not shown). A display 128 (e.g., a screen or a display monitor) is connected to the system bus 104 by the display adapter 124, which may include a graphics controller to improve the performance of graphics intensive applications and a video controller. A keyboard 130, a mouse 132, among other input/output devices, can be interconnected to the system bus 104 via the interface adapter 126, which may include, for example, a Super I/O chip integrating multiple device adapters into a single integrated circuit. Suitable I/O buses for connecting peripheral devices such as hard disk controllers, network adapters, and graphics adapters typically include common protocols, such as the Peripheral Component Interconnect (PCI). Thus, as configured in FIG. 1, the computing system 100 includes processing capability in the form of the processors 102, and, storage capability including the system memory 106 and the mass storage 118, input means such as the keyboard 130 and the mouse 132, and output capability including the display 128.

In some embodiments, the communications adapter 114 can transmit data using any suitable interface or protocol, such as the internet small computing system interface, among others. The network 122 may be a cellular network, a radio network, a wide area network (WAN), a local area network (LAN), or the Internet, among others. An external computing device may connect to the computing system 100 through the network 122. In some examples, an external computing device may be an external webserver or a cloud computing node.

It is to be understood that the block diagram of FIG. 1 is not intended to indicate that the computing system 100 is to include all of the components shown in FIG. 1. Rather, the computing system 100 can include any appropriate fewer or additional components not illustrated in FIG. 1 (e.g., additional memory components, embedded controllers, modules, additional network interfaces, etc.). Further, the embodiments described herein with respect to computing system 100 may be implemented with any appropriate logic, wherein the logic, as referred to herein, can include any suitable hardware (e.g., a processor, an embedded controller, or an application specific integrated circuit, among others), software (e.g., an application, among others), firmware, or any suitable combination of hardware, software, and firmware, in various embodiments.

FIG. 2 illustrates a method 200 for predicting a fatigue response of a material according to an aspect of the present disclosure. The method 200 may be implemented, for example, in conjunction with the computing system 100 illustrated in FIG. 1. Block 202 of the method 200 involves receiving a user input specifying values of one or more surface roughness parameters. Herein, the user may provide input on one or more roughness parameters that are typically used to characterize the surface of the material for which fatigue life is to be predicted. The one or more parameters may be selected, for example, from the non-limiting examples of surface roughness parameters provided below. It is to be appreciated that the disclosed techniques may be used to account for additional or different parameters.

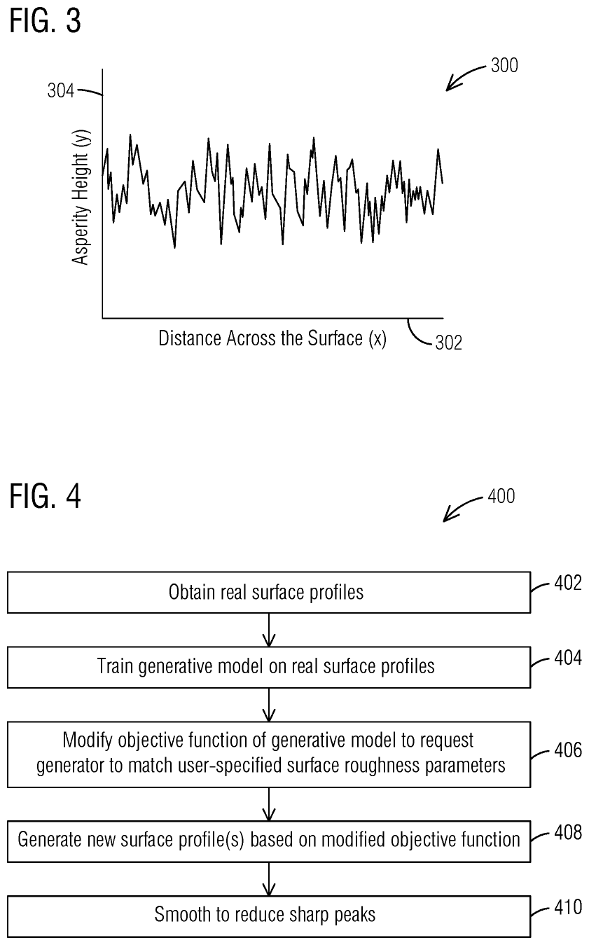

FIG. 3 shows an illustrative one-dimensional (1D) surface curve 300 defining a surface having microscopic asperities, where the axis 302 represents a distance across the surface (x) and the axis y represents asperity height (y). Assuming the profile is represented as a set of (xᵢ, yᵢ) points, the surface roughness parameters can be defined as:
Arithmetical mean deviation:

$${R}_{a} = \frac{1}{n} {\sum }_{i = 1}^{n} \vert {y}_{i} \vert , ,$$

where yᵢ is the height value at a single point of a 1D surface profile;
Root mean squared:

$${R}_{q} = \sqrt{\frac{1}{n} {\sum }_{i = 1}^{n} {y}_{i}^{2}}$$

Maximum valley depth:

$${R}_{v} = {min}_{i} {y}_{i}$$

Maximum peak height:

$${R}_{p} = {max}_{i} {y}_{i}$$

Maximum height of the profile: Rₜ=Rₚ−Rᵥ
Average maximum valley depth:

$${R}_{v m} = \frac{1}{5} {\sum }_{i = 1}^{5} \vert {R}_{v i} \vert$$

Average maximum peak height:

$${R}_{p m} = \frac{1}{5} {\sum }_{i = 1}^{5} \vert {R}_{p i} \vert$$

Skewness:

$${R}_{s k} = \frac{1}{n {R}_{q}^{3}} {\sum }_{i = 1}^{n} {y}_{i}^{3}$$

Kurtosis:

$${R}_{k u} = \frac{1}{n {R}_{q}^{4}} {\sum }_{i = 1}^{n} {y}_{i}^{4}$$

Referring back to FIG. 2, block 204 of the method 200 involves generating at least one realistic virtual surface profile from the specified one or more surface roughness parameters. In the context of the present specification, a “virtual surface profile” refers to a machine generated surface profile obtained from user-specified surface roughness parameter values using a data-driven generative method. In this regard, a virtual surface profile is an artificial surface profile, and is not to be construed as being generated from surface measurements of a real surface. However, a virtual surface may be generated to closely resemble real surface profiles in an available data set. Accordingly, such a virtual surface profile is also described as being “realistic”, while not being real. Example embodiments data-driven approaches for generating virtual surface profiles are illustrated below referring to FIG. 4-6. Continuing with reference to FIG. 2, block 206 of the method 200 involves predicting fatigue life of the material based on a stress field applied to the generated virtual surface profile. This may comprise generating a stress-life (S-N curve) plotting cyclic stress amplitude S against fatigue life N_{f} defined by number of cycles to failure. The generated S-N curve may be graphically displayed on a display screen. Exemplary non-limiting implementations within the scope of the above-described method 200 are illustrated below referring to FIG. 7-9.

FIG. 4 illustrates a first example method 400 for generating one or more virtual surface profiles based on the user-specified surface roughness parameters. The method 400 utilizes a machine learning based generative model. Examples of such models include Generative Adversarial Networks (GAN), Variational Auto-Encoders, or their variations (e.g. WGAN-GP), among others. The generative model may be trained on the available data and utilized to generate novel virtual surface profiles that resemble the surface profiles in the available data. In the shown example, the method 400 utilizes a GAN.

Block 402 of the method 400 involves obtaining, as training data, 1D surface profiles s from surface measurements and calculated roughness parameters r of a plurality of real surfaces. The data from real surfaces may be pre-processed, which may, for example, involve the steps of resampling the data to obtain same sampling rate throughout all the samples, splitting the data to obtain the same sample length, detrending, etc. Block 404 of the method 400 involves training the generative model using the training data obtained at block 402. At block 406, the generative model is modified to request to the generator to match one or more of the surface roughness parameters specified by the user. In the case of GAN, this can be achieved, for example, by modifying the objective function that the process optimizes by adding the following term:

Σᵢ₌₁⁹E{[Rᵢ(\tilde{s})−Rᵢ(s)]²}

In the above term, \tilde{s}=G(z, r) are the generated 1D surface profiles, as function of the generator G, z is latent vector and Rᵢ(*) is the function that represents the roughness parameters i. The objective function E is summed over the number of surface roughness parameters specified, which in this case is 9.

Block 408 of the method 400 involves the generation of a specified number of new (virtual) 1D surface profiles \tilde{s}, that closely resemble the surface profiles s in the training data, based on the modified objective function of the generative model. As an additional step, at block 410, the generated virtual 1D surface profile may be smoothed to reduce sharp peaks, for example, using spectral or spline interpolation.

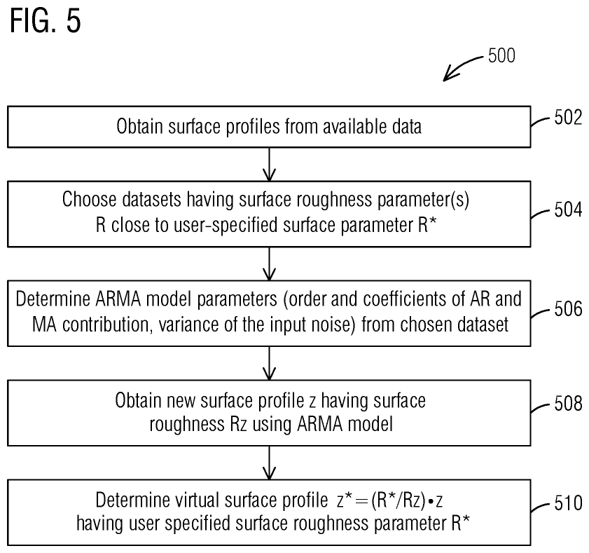

FIG. 5 illustrates a second example method 500 for generating one or more virtual surface profiles based on the user-specified surface roughness parameters. The method 500 uses an Autoregressive Moving Average (ARMA) model to generate new surface profiles. The basic principle of this approach is to identify a suitable mathematical model describing the surface height profile from the available data and using the same model to generate new surface profiles.

Block 502 of the method 500 involves obtaining surface profiles from available data. The available data comprises surface profiles obtained from surface measurements and calculated roughness parameters of a plurality of real surfaces. The data from real surfaces may be pre-processed, which may, for example, involve the steps of resampling the data to obtain same sampling rate throughout all the samples, splitting the data to obtain the same sample length, detrending, etc. Block 504 of the method 500 involves choosing datasets having surface roughness parameters R that are already close to the user-specified surface roughness parameters R*.

Block 506 of the method of the method 500 involves determining a mathematical model of the following form from the chosen dataset.

yₜ=Σᵢ₌₁^{p}aᵢyₜ₋ᵢ+Σⱼ₌₁^{q}bⱼϵₜ₋ⱼ+ϵₜ

In the above model, yₜ denotes the surface height at spatial position xₜ and the coefficients aₜ and bⱼ determine the autoregressive (AR) and moving average (MA) part of the model, respectively. Furthermore, the quantity ϵₜ denotes white noise, i.e. a random input with mean zero and fixed variance σ², at position xₜ.

The following model parameters are determined from the chosen dataset, namely order of the AR and MA contribution p, q, coefficients of the AR and MA contribution a₁, . . . , aₚ, b₁, . . . , b_{q} and variation of the input noise σ². For each dataset, these quantities may be identified using, for example, a Box-Jenkins approach in combination with a minimization of Akaike's Information Criterion (AIC). The Box-Jenkins approach is discussed in the publication: Box, George, & Jenkins, Gwilym (1970). Time Series Analysis: Forecasting and Control. San Francisco: Holden-Day. The Akaike's Information Criterion (AIC) is discussed in the publication: Hyndman, R. J., & Athanasopoulos, G. (2018) Forecasting: principles and practice, 2nd edition, OTexts: Melbourne, Australia. OTexts.com/fpp2. Accessed on Aug. 29, 2019.

Under the assumption that there is a single model describing all surface profiles, the aforementioned model parameters may be obtained by averaging the corresponding results from all datasets. The result is an ARMA model describing the spatial evolution of the surface height profile.

Block 508 of the method 500 involves obtaining one or more new surface profiles z using the ARMA model developed in block 506. The new surface profile z may be obtained by specifying an initial height at x₀=0 (for simplicity this can be assumed to be zero) and creating a series of random white noise input ϵₜ with variance σ². As would be expected, the new surface profile z would have surface roughness parameter parameters Rz, where Rz≈R*. Finally, at block 510 of the method 500, a virtual surface z* is generated which would have the user-specified surface roughness parameter R*. The surface profile z* is determined as z*=(R*/Rz)·z.

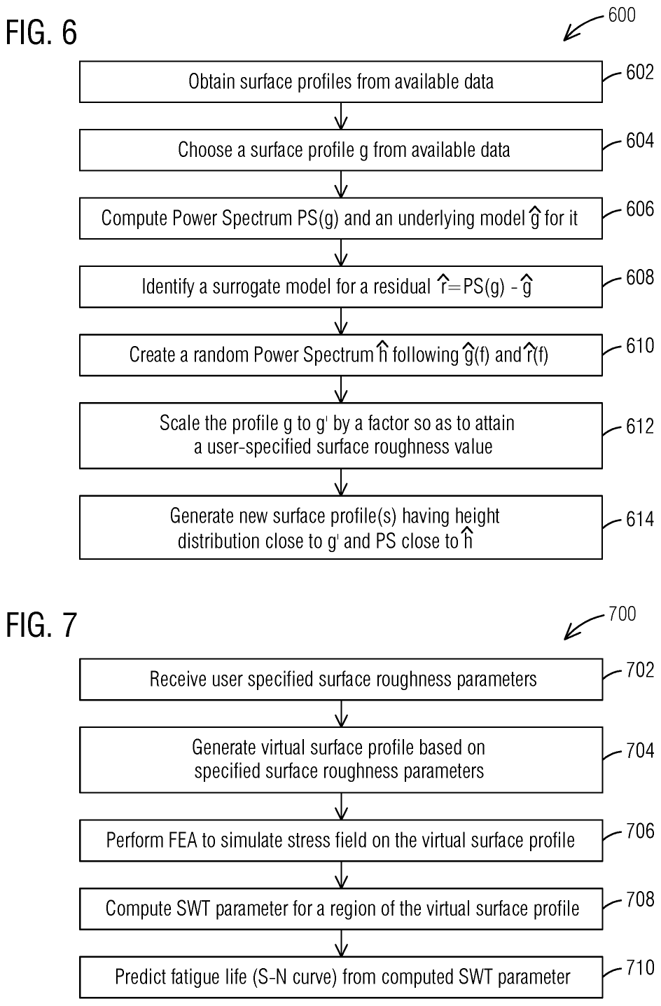

FIG. 6 illustrates a third example method 600 for generating one or more virtual surface profiles based on the user-specified surface roughness parameters. While the method 500 shown in FIG. 5 works directly on the height data of the surface profiles, the method 600 shown in FIG. 6 identifies typical surface height frequency patterns from available data and creating new (virtual) surface profiles based on those patterns.

Block 602 of the example method 600 involves obtaining surface profiles from available data. The available data comprises surface profiles obtained from surface measurements and calculated roughness parameters of a plurality of real surfaces. The data from real surfaces may be pre-processed, which may, for example, involve the steps of resampling the data to obtain same sampling rate throughout all the samples, splitting the data to obtain the same sample length, detrending, etc. Block 604 of the method 600 involves choosing a surface profile g from the available data. A particular strategy for the choice is presented at block 612. Block 606 of the method 600 involves computing a Power Spectrum (PS) of g, namely, PS(g), and an underlying model ĝ for it via the nonlinear least squares (NLS) method. It has been observed that the following parameterized model presents a good candidate.

ĝ(f)=(af³+bf²+cf)·exp(−βf)

In the above model, the parameters a, b, c, β may be identified by NLS.

Block 608 of the method 600 involves identifying a surrogate model for the residual or noise term \hat{r}=PS(g)−ĝ, again via NLS. Typically, \hat{r} is normally distributed with variance decaying exponentially for increasing frequencies. This is due to the fact that such frequencies larger than a certain threshold value are essentially non-existent. This decay motivates a model of the form

\hat{r}(f)˜N(0,σ_{f}(f)²)

In the above model σ_{f}(f)=γ·exp(−δ·f). It can be seen that the variance decreases if the frequency f increases.

Block 610 of the method 600 involves creating a random Power Spectrum ĥ following the trend ĝ(f) and a noise term \hat{r}(f) from above-mentioned distribution. The generated surface profile will eventually have a PS close to ĥ.

Block 612 of the method 600 involves scaling the profile g by a factor ω such that g′=ω·g attains a desired surface roughness value as specified by the user. In order to minimize the distortion in the data, in block 604, the surface g should be chosen as the one having a roughness value that is closest to the desired surface roughness among all datasets.

Block 614 of the method 600 involves generating one or more new (virtual) surface profiles such that the new surface profile has (a) the same height distribution and hence the same surface roughness as g′ and (b) a Power Spectrum that is very close to ĥ and is therefore assumed to be realistic as ĥ was identified from the data. The new surface profile(s) may be generated using an iterative version of the amplitude adjusted Fourier transform (AAFT). A discussion on AAFT is available in the publication: Schreiber, Thomas, & Andreas Schmitz. “Improved surrogate data for nonlinearity tests.” Physical Review Letters 77.4 (1996): 635.

One or more of the methods illustrated in FIG. 4-6 may be used, individually, or in combination, to generate new (virtual) surface profiles. For example, the methods of FIGS. 4 and 6 may be combined to achieve machine learning generated models that resemble the training data and also achieve the frequency patterns extracted from the available data. A first way to accomplish the above is to proceed with the method 400 shown in FIG. 4, and then (after block 410) add additional blocks as described for the method 600 of FIG. 6. A second way to accomplish the above is to add a term in the objective function of the generative model (see block 406 of FIG. 4) to penalize in the generative process surface profiles whose frequency content differ from the desired one.

Referring now to FIG. 7, a first example implementation is described of a method 700 for predicting fatigue response of a material based on surface roughness. At block 702 of the method 700, a user input is received specifying values of one or more surface roughness parameters characterizing a surface of the material for which fatigue life is to be predicted. The specified surface roughness parameters may include one or more of the surface roughness parameters described above, or may include additional or different surface roughness parameters. Block 704 of the method 700 involves generating at least one virtual surface profile based on the surface roughness parameters specified in block 702. The virtual surface profile may be generated using one or more of the techniques illustrated above referring to FIG. 4-6.

At block 706, the method 700 involves creating a simulation model using finite element analysis (FEA), based on the generated virtual surface profile. The method includes applying appropriate loads and constraints to the FEA model to simulate a stress field on the virtual surface. In some embodiments, the simulated stress field may be graphically displayed on a display screen. While at a global level, the material may be assumed to exhibit a linear elastic behavior, at a local level, the material may exhibit plastic behavior due to stress concentration effects caused by surface roughness, porosity, etc. Accordingly, at block 708, the method 700 computes a Smith-Watson-Topper (SWT) parameter to account for the localized plastic behavior, in order to predict fatigue life of the material. The SWT parameter, is known in the art (see Smith, K. N., Watson, P. and Topper, T. H. (1970) A stress-strain function for the fatigue of materials. J. Mater. 5, 767-778.), and is defined by the following equation:

$$\frac{{Δ}_{ε}}{2} {σ}_{max} = \frac{{( {σ}_{f}^{′} )}^{l}}{E} {( 2 {N}_{f} )}^{2 b} + {σ}_{f}^{′} {{ε}_{f}^{′} ( 2 {N}_{f} )}^{b + c}$$

In the above equation, the term on the left is the SWT parameter, which is a product of the maximum stress σₘₐₓ and the strain value Δε/2 for that said maximum stress. In the expression on the right, N_{f} denotes fatigue life defined by number of cycles to failure, b is the Basquin exponent, c is the Coffin-Mansion exponent, and σ′_{f}, E and ε′_{f} are constants. The values σₘₐₓ and Δε/2 may be computed from the FEA model, to thereby determine a value of the SWT parameter for a region of the virtual surface profile, which may be then used to determine fatigue life N_{f} based on the above equation.

Finally, at block 710 of the method 700, a fatigue life of the material is predicted based on the computed SWT parameter. This may comprise generating an S-N curve plotting cyclic stress amplitude S against fatigue life N_{f} defined by number of cycles to failure. The generated S-N curve may be graphically displayed on a display screen. The computed SWT parameters may be related to the S-N curve through the well-known Basquin-Coffin-Mansion equation.

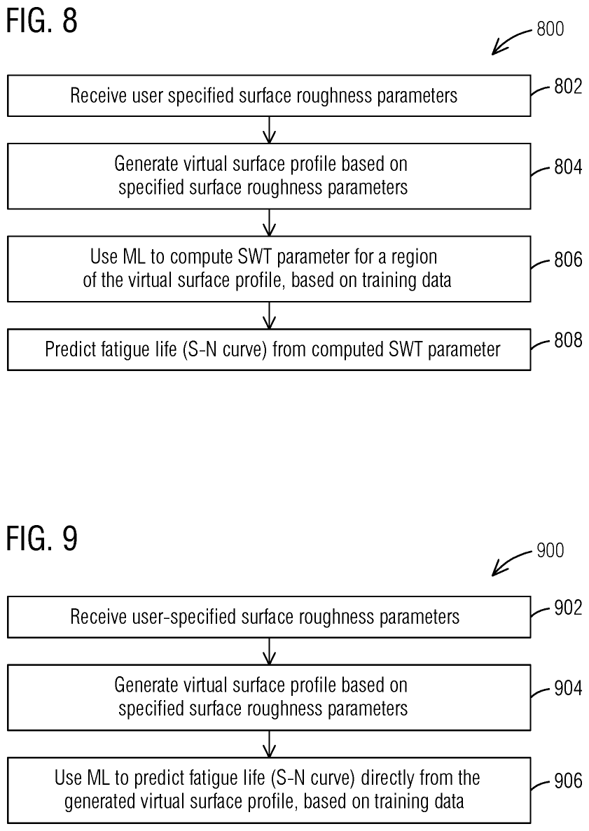

FIG. 8 illustrates a second example implementation of a method 800 for predicting fatigue response of a material based on surface roughness. The proposed method 800 uses a combination of machine learning (ML) and FEA based simulation to speed up the computation of the SWT parameter in order to predict fatigue life.

At block 802 of the method 800, a user input is received specifying values of one or more surface roughness parameters characterizing a surface of the material for which fatigue life is to be predicted. The specified surface roughness parameters may include one or more of the surface roughness parameters described above, or may include additional or different surface roughness parameters. Block 804 of the method 800 involves generating at least one virtual surface profile based on the surface roughness parameters specified in block 802. The virtual surface profile may be generated using one or more of the techniques illustrated above referring to FIG. 4-6.

At block 806 of the method 800, a value of the SWT parameter for a region of the virtual surface profile is determined using an ML based model. The ML based model is trained on previously generated data points pertaining to a large number of sample virtual surface profiles. Each training data point is generated by simulating a stress field on a respective sample virtual surface profile using FEA, and computing therefrom an SWT parameter for a region of the sample virtual surface profile, similar to that described in connection with blocks 706 and 708 of FIG. 7. The ML based model of FIG. 8 may be thus trained to map the input surface profile to the result of the finite element simulation (i.e., the computed value of the SWT parameter). To this end, the method 700 of FIG. 7 may be utilized for generating the training data points for implementing the method 800 of FIG. 8. Once a sufficiently large number of data points are generated using the method 700, the method 800 may be implemented, to accelerate the computation of the SWT parameter.

At block 808 of the method 800, a fatigue life of the material is predicted based on the computed SWT parameter. This may comprise generating an S-N curve plotting cyclic stress amplitude S against fatigue life N_{f} defined by number of cycles to failure. The generated S-N curve may be graphically displayed on a display screen. The computed SWT parameters may be related to the S-N curve through the well-known Basquin-Coffin-Mansion equation. In case of the method 800, the determination of the predicted fatigue life is greatly simplified by computing the SWT parameter directly based on ML, bypassing the more time-consuming FEA based approach.

FIG. 9 illustrates a third example implementation of a method 900 for predicting fatigue response of a material based on surface roughness. The proposed method 900 utilizes ML to predict fatigue life directly from the generated virtual surface profile.

At block 902 of the method 900, a user input is received specifying values of one or more surface roughness parameters characterizing a surface of the material for which fatigue life is to be predicted. The specified surface roughness parameters may include one or more of the surface roughness parameters described above, or may include additional or different surface roughness parameters. Block 904 of the method 900 involves generating at least one virtual surface profile based on the surface roughness parameters specified in block 902. The virtual surface profile may be generated using one or more of the techniques illustrated above referring to FIG. 4-6.

At block 906 of the method 900, the fatigue life of the material is predicted (and may also be graphically displayed on a display screen), for example, in the form of an S-N curve, directly from the generated virtual surface profile, using the ML based model. The ML based model is trained on previously generated data points pertaining to a large number of sample virtual surface profiles. Each training data point is generated by simulating a stress field on a respective sample virtual surface profile using FEA, and computing therefrom an SWT parameter for a region of the sample virtual surface profile, and determining fatigue life (S-N curve) corresponding to the sample virtual surface profile from the computed SWT parameter. The ML based model of FIG. 9 may be thus trained to map the input surface profile to the predicted S-N curve.

To generate the training data points for implementing the method 900, the method 700 (FIG. 7) and the method 800 (FIG. 8) may be utilized. The implementation of the method 900 is based on the understanding that the simulated stress fields contain enough information to be able to predict when a component would fail. However, this might involve a large amount of data for the ML based model to process and learn patterns from. Accordingly, the method 900 may be implemented only after a sufficiently large number of data points are generated linking the stress fields to the eventual S-N curves. The methods 700 and 800 may be utilized to allow the ML based model to reach to the required level of learning.

Aspects of the present disclosure may include a system, a method, and/or a computer program product at any possible technical detail level of integration. The computer program product may include a computer readable storage medium (or media) having computer readable program instructions thereon for causing a processor to carry out aspects of the present disclosure. A computer readable storage medium, as used herein, is understood to be a non-transitory storage medium, which is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The functions and process steps herein may be performed automatically, wholly or partially in response to user command. An activity (including a step) performed automatically is performed in response to one or more executable instructions or device operation without user direct initiation of the activity.

The system and processes of the figures are not exclusive. Other systems and processes may be derived in accordance with the principles of the disclosure to accomplish the same objectives. Although this disclosure has been described with reference to particular embodiments, it is to be understood that the embodiments and variations shown and described herein are for illustration purposes only. Modifications to the current design may be implemented by those skilled in the art, without departing from the scope of the disclosure.

1. A computer-implemented method for predicting a fatigue response of a material, comprising:
receiving a user input specifying one or more surface roughness parameters that characterize a surface of a material for which fatigue life is to be predicted,
generating at least one realistic virtual surface profile from the specified one or more surface roughness parameters, and
predicting fatigue life of the material in dependence of a stress field applied to the generated virtual surface profile.

2. The method according to claim 1, wherein the at least one realistic virtual surface profile is generated by a machine learning based generative model, the machine learning based generative model being developed using training data comprising surface profiles obtained from surface measurements and calculated roughness parameters of a plurality of real surfaces.

3. The method according to claim 1, wherein the at least one virtual surface profile is generated by an Autoregressive Moving Average (ARMA) model, the ARMA model being developed to describe a surface height profile from available data comprising surface profiles obtained from surface measurements and calculated roughness parameters of a plurality of real surfaces.

4. The method according to claim 1, wherein the at least one virtual surface profile is generated based on surface height frequency patterns identified from available data comprising surface profiles obtained from surface measurements and calculated roughness parameters of a plurality of real surfaces.

5. The method according to claim 1, wherein the prediction of the fatigue life of the material is based on a computation of a Smith-Watson-Topper (SWT) parameter from the applied stress field on the virtual surface profile.

6. The method according to claim 5, wherein the computation of the SWT parameter comprises performing a finite element analysis to generate a simulated stress field on the virtual surface profile, and determining a value of the SWT parameter for a region of the virtual surface profile.

7. The method according to claim 5, wherein the computation of the SWT parameter comprises determining a value of the SWT parameter for a region of the virtual surface profile using a machine learning based model,
wherein the machine learning based model is trained on data points pertaining to a plurality of sample virtual surface profiles, each training data point being generated by performing finite element analysis to simulate a stress field on a respective sample virtual surface profile, and compute therefrom an SWT parameter for a region of the sample virtual surface profile.

8. The method according to claim 5, wherein predicting the fatigue life of the material comprises generating a stress-life (S-N) curve from the computed SWT parameter.

9. The method according to claim 1, wherein the fatigue life of the material is predicted based on a machine learning based model trained to predict fatigue life directly from the generated virtual surface profile,
wherein the machine learning based model is trained on data points pertaining to a plurality of sample virtual surface profiles, each training data point being generated by:
performing finite element analysis to simulate a stress field on a respective sample virtual surface profile, and compute therefrom a Smith-Watson-Topper (SWT) parameter for a region of the sample virtual surface profile, and
determining fatigue life corresponding to the sample virtual surface profile from the computed SWT parameter.

10. The method according to claim 9, predicting the fatigue life of the material comprises generating a stress-life (S-N) curve using the machine learning based model.

11. A non-transitory computer-readable storage medium including instructions that, when processed by a computer, configure the computer to perform the method according to claim 1.

12. A computing system comprising:
a processor; and
a memory storing instructions that, when executed by the processor, configure the computing system to:
receive a user input specifying one or more surface roughness parameters that characterize a surface of a material for which fatigue life is to be predicted,
generate at least one realistic virtual surface profile from the specified one or more surface roughness parameters, and
predict fatigue life of the material in dependence of a stress field applied to the generated virtual surface profile.

13. The computing system according to claim 12, wherein the at least one realistic virtual surface profile is generated by a machine learning based generative model, the machine learning based generative model being developed using training data comprising surface profiles obtained from surface measurements and calculated roughness parameters of a plurality of real surfaces.

14. The computing system according to claim 12, wherein the at least one virtual surface profile is generated by an Autoregressive Moving Average (ARMA) model, the ARMA model being developed to describe a surface height profile from available data comprising surface profiles obtained from surface measurements and calculated roughness parameters of a plurality of real surfaces.

15. The computing system according to claim 12, wherein the at least one virtual surface profile is generated based on surface height frequency patterns identified from available data comprising surface profiles obtained from surface measurements and calculated roughness parameters of a plurality of real surfaces.

16. The computing system according to claim 12, wherein the prediction of the fatigue life of the material is based on a computation of a Smith-Watson-Topper (SWT) parameter from the applied stress field on the virtual surface profile.

17. The computing system according to claim 16, wherein the computation of the SWT parameter comprises performing a finite element analysis to generate a simulated stress field on the virtual surface profile, and determining a value of the SWT parameter for a region of the virtual surface profile.

18. The computing system according to claim 16, wherein the computation of the SWT parameter comprises determining a value of the SWT parameter for a region of the virtual surface profile based on a machine learning based model,
wherein the machine learning based model is trained on data points pertaining to a plurality of sample virtual surface profiles, each training data point being generated by performing finite element analysis to simulate a stress field on a respective sample virtual surface profile, and compute therefrom an SWT parameter for a region of the sample virtual surface profile.

19. The computing system according to claim 16, wherein predicting the fatigue life of the material comprises generating a stress-life (S-N) curve from the computed SWT parameter.

20. The computing system according to claim 12, wherein the fatigue life of the material is predicted based on a machine learning based model trained to predict fatigue life directly from the generated virtual surface profile,
wherein the machine learning based model is trained on data points pertaining to a plurality of sample virtual surface profiles, each training data point being generated by:
performing finite element analysis to simulate a stress field on a respective sample virtual surface profile, and compute therefrom a Smith-Watson-Topper (SWT) parameter for a region of the sample virtual surface profile, and
determining fatigue life corresponding to the sample virtual surface profile from the computed SWT parameter.
